(19) United States
(12) Patent Application Publication
(10) Pub. No.: US 2007/0173819 A1
(43) Pub. Date: Jul. 26, 2007

(54) Spinal implant fixation assembly

(76) Inventor: Robin Sandlin, Memphis, TN (US)

Correspondence Address: Amy E. Rinaldo;KOHN & ASSOCIATES, PLLC, Suite 410, 30500 Northwestern Highway, Farmington Hills, MI, 48334, US

(21) Appl. No.: 11/329,644

(22) Filed: Jan. 11, 2006

(51) Int. Cl. A61F 2/30

(52) U.S. Cl. 606/61

(57) ABSTRACT

A locking mechanism that is a non-threaded locking device for locking a rod in place within a screw and rod fixation assembly.

### BACKGROUND OF THE INVENTION

1. Technical Field

The present invention relates to fixation assemblies. More particularly, the present invention relates to locking caps for use in spinal implant fixation assemblies.

2. Background Art

Several techniques and systems have been developed for correcting and stabilizing the spine and for facilitating fusion at various levels of the spine. Stabilization of the spine for various conditions, including degenerative disk disease, scoliosis, spondylolisthesis, and spinal stenosis, often require attaching implants to the spine and then securing the implants to spinal rods. Such spinal fixation devices can immobilize the vertebrae of the spine and can alter the alignment of the spine over a large number of vertebrae by connecting at least one elongate rod to the sequence of selected vertebrae. These rods can span a large number of vertebrae, such as three or four. The spine anatomy, however, rarely allows for three or more implants to be directly in line. In order to allow for this irregularity, the rod must be contoured to the coronal plane.

Spinal fixation has become a common approach in fusion of vertebrae and treating fractures and the above listed spinal disorders. A common device used for spinal fixation is a bone fixation plate assembly. Typical bone fixation plate assemblies have a relatively flat, rectangular plate with a plurality of apertures therethrough. Additional assemblies include an implantation fixation system that locks a rod to several vertebrae. In these assemblies, as in with other spinal fixation systems, they utilize various fasteners, such as bone screws, to secure the bone fixation plate assembly or the implantation fixation assembly to the desired and targeted vertebrae of the patient. These screws vary in design and shape depending upon their desired location and use thereof.

In particular, polyaxial locking screws are used with these devices. The key to the polyaxial screws used with these systems is having the screw head being securely fastened to the vertebrae and to the assembly thereof. Thus, the polyaxial screws must be used in conjunction with a type of screw head securing device that provides a strong lock to the polyaxial screw. Any movement of the screw can be detrimental towards the healing process of the spine. Further, additional damage can occur if there is movement of the screw once it has been fixed to the vertebrae. Therefore, movement of the screw must be minimized or eliminated.

There are numerous polyaxial screws existing in the market today and known in the prior art. Additionally, numerous devices exist that provide a securing means for locking the polyaxial screw. For example, U.S. Pat. Nos. 5,554,157, 5,549,608, and 5,586,984 all to Errico et al. disclose polyaxial locking screws and coupling element devices for use with a rod fixation apparatus. The '157 Patent discloses a coupling element including an interior axial passage having an interior surface that is inwardly curvate at the lower portion thereof such that it comprises a socket for polyaxially retaining a spherical head of a screw. The coupling element further includes a pair of vertically oriented opposing channels extending down from the top of the coupling element, which define therebetween a rod receiving seat. The channel further provides for walls of the upper portion to a pair of upwardly extending members, each including an exterior threading disposed on the uppermost portion thereof for receiving a locking nut. During the implantation of the assembly, the locking nut seals against the top of the rod that in turn seats on top of the screw head. The nut causes the rod to be locked between the nut and the screw and the screw to be locked in the socket.

The '608 Patent discloses a modification wherein a locking ring is disposed about the exterior of the lower portion of the coupling element and provides an inward force on an outwardly tapered portion upon downward translation thereof. As a result, the interior chamber crush locks a screw head therein to eliminate the polyaxial nature of the screw element coupling.

The '984 Patent discloses a polyaxial orthopedic device including a coupling element having a tapered lower portion having a slotted interior chamber in which a curvate head of a screw is initially polyaxially disposed. The coupling element includes a recess for receiving a rod of the implant apparatus. A locking ring is disposed about the lower portion of the coupling element and provides an inward force on the outwardly tapered portion upon downward translation thereof. The vertical slots are caused to close and crush, thereby locking the screw head within the interior chamber thereof.

U.S. Pat. No. 6,280,442 to Barker et al. discloses a complex locking mechanism having a screw head with a complex head geometry, a crown member, and an outer rigid body. Locking occurs by compressing the crown member against the complex head, which compresses the head against the rigid seat. This compression crushes the machine ridges on the head and secures the screw therein.

Another example of a common locking mechanism is a type of collet that has a spherical seat with a flexible portion that is designed to deflect around the screw. By compressing the flexible portion against a rigid, outer wall, the collet is compressed against the head to cause locking therein. Examples of these collets are found in numerous patents. For example, U.S. Pat. No. 6,053,917 to Sherman et al. discloses a multiaxial bone screw assembly that includes a bone screw having a partially spherical head. Additionally, the assembly includes a receiver member that has a central bore that defines a tapered recess to receive a contracting collet carrying the head of the bone screw. The collet defines a partially spherical recess to receive the head of the bone screw and includes deflectable fingers that substantially surround the screw head. As a set screw is tightened into the receiver member, the set screw compresses the rod against the collet, which presses the collet into the tapered recess of the receiver member, thereby deflecting the fingers of the collet against the bone screw head.

Another patent, U.S. Pat. No. 5,964,760 to Richelsoph, discloses a spinal implant fixation assembly that includes a bone fixation member. A rod receiving seat is operatively connected to the bone fixation element for seating a portion of a rod therein. A locking mechanism, in the form of a nut and locking ring, engages the rod receiving seat for forcing an inner wall of the rod receiving seat to contour around and engage the rod seated therein and for locking and fixing the rod relative to the inner housing. The assembly further includes a screw head receiving insert for obtaining a head of a screw therein. The insert is moveable within the assembly between a locked position entrapping the screw head and an unlocked position wherein the screw head enters or escapes.

Other polyaxial screw patents that utilize a similar collet are disclosed in U.S. Pat. No. 6,010,503 to Richelsoph, U.S. Pat. No. 5,910,142 to Tatar (disclosing the use of a spherical collet that is compressed between the screw head and the rod), and U.S. Pat. No. 5,891,145 to Morrison et al. (disclosing the use of a very complex double wedge locking mechanism).

More specifically, the '142 Patent to Tatar discloses a polyaxial pedicle screw device for use with a rod implant apparatus, which utilizes a rod mounted ferrule. The device further includes a screw having a curvate head and a rod receiving body. The body has a rod receiving channel and an axial bore into which the head of the screw is inserted. The rod mounted ferrule seats into a small curvate recess in the upper portion of the screw head such that the rod may enter the body at a variety of angles while maintaining secure seating against the head of the screw. The insertion of a top, set-screw compresses down on the ferrule, locking the rod in position and onto the screw head. Further, the body is locked in position to completely secure the assembly.

The '145 Patent to Morrison et al. discloses a spinal fixation assembly that includes a bone engaging fastener and an elongated member such as a spinal rod. The fixation assembly is a multiaxial assembly that permits fixation of the bone engaging fastener to the spinal rod at any of the continuous ranges of angles relative to the rod in three dimensional space. The fixation assembly includes a receiver member having a bore therethrough, the walls of which are tapered near the bottom, and a channel communicating with the bore and having an upper opening at the top of the receiver member for insertion of a spinal rod. An outer wedge member and an inner wedge member are also included. Both members have the general shape of a washer and a bore therethrough. In each wedge member, the respective bore is not parallel to the central axis of the respective wedge member. Additionally, the outside surfaces of the wedge members may be tapered and the respective bores may be tapered so as to self-lock when seated and tightened. The bone engaging fastener fits within the bore of the inner wedge member, which in turn fits within the bore of the outer wedge member, which in turn fits within the tapered sides of the receiver member. When the desired position of the bone engaging fastener in three dimensional space is attained, the components are seated to achieve a tight friction fit.

U.S. Pat. No. 6,063,089 to Errico et al. discloses a polyaxial orthopedic device for use with a rod implant apparatus that includes a screw having a head, a tubular body having holes on top, side, and bottom thereof, and a rod coupling element. The head of the screw is disposed in the body with the shaft of the screw extending out of the bottom hole, such that the body and the screw may initially rotate relative to one another. The rod coupling element has a ball shaped end that sits in the body with the remainder of the rod coupling element extending out of the side hole of the body, such that the rod coupling element and the body are initially polyaxially coupled relative to one another. The ball end of the rod coupling element is disposed on top of the head of the screw. A set screw is provided on top of the body, the tightening of which causes the ball, head, and body to be crushed locked together, thereby preventing further relative motion.

In all of the existing prior art, particularly those described herein, the polyaxial screws utilize a complex locking mechanism and additional locking parts to prevent the movement of the polyaxial screw. Typically, the more complex the locking mechanism, larger components are needed and manufacturing costs are expensive. Locking and achieving strong hold values become more difficult with more parts. Further, the sizes of the various fixation plates and fixation assemblies are critical to these types of surgeries. Bulky components can cause soft tissue irritation, as well as compromise the facet joints at the end of a fusion. Minimizing the size of the implants used is critical in spinal surgery. Soft tissue irritation resulting from extensions of implants is a common occurrence. Many times, it is caused by the implant being thick relative to its environment. For example, implants can be too thick to be sufficiently covered within the muscle tissue. Hence, a reduction in the overall thickness of the implant is a critical advantage.

Accordingly, there is a need for a screw head securing mechanism or device that provides a strong, effective, and secure lock of the screw head in its desired position. Additionally, there is a need for a screw head securing mechanism or device that is minimal in size and has a reduced amount of components to provide for a simpler, more effective, and less cumbersome device for fixing screws.

### SUMMARY OF THE INVENTION

According to the present invention, there is provided a locking mechanism that is a non-threaded locking device for locking a rod in place within a screw and rod fixation assembly.

### DESCRIPTION OF THE DRAWINGS

Other advantages of the present invention are readily appreciated as the same becomes better understood by reference to the following detailed description when considered in connection with the accompanying drawings wherein:

FIG. 1 is a side view partially in cross section of a first embodiment of the present invention;

FIG. 2 is a side view of a second embodiment of the present invention;

FIG. 3 is a side exploded view of a third embodiment of the present invention; and

FIG. 4 is a cross sectional side view of the third embodiment of the present invention as assembled.

FIG. 5 is a cross sectional view of an embodiment of the screw and rod fixation assembly wherein the locking mechanism is a non-threaded tapered cap;

FIG. 6 is a cross sectional view of an embodiment of the screw and rod fixation assembly wherein the locking mechanism is a non-threaded, tapered, and grooved cap;

FIG. 7 is a side view of the device for inserting the cap of the present invention;

FIG. 8 is a cross sectional view of a further embodiment of the present invention;

FIG. 9 is a cross sectional view of the forth embodiment of the present invention and a screw member disposed adjacent the assembly;

FIG. 10 shows the screw member inserted into the pocket of the forth embodiment of the present invention;

FIG. 11 shows the screw member and a rod member locked within the assembly, the assembly being shown in cross section;

FIG. 12 shows a side view, and cross section of the insert member;

FIG. 13 shows a cross sectional view of the body portion of the forth embodiment of the present invention;

FIG. 14 shows a cross sectional view of the assembly having straight rod disposed therein;

FIG. 15 shows a cross section of the assembly having a curved rod disposed therein;

FIG. 16 is side view of the insert member of the forth embodiment of the present invention;

FIG. 17 shows a side view of a second embodiment of the insert member;

FIG. 18 shows a cross sectional view of a second embodiment of the body portion of the forth embodiment;

FIG. 19 is a side view of a third embodiment of the insert;

FIG. 20 is a side view of a forth embodiment of the insert combining the embodiment of the insert combining the embodiment shown in FIGS. 17 and 19; and

FIG. 21 is a cross sectional view of the third embodiment of the body portion.

### DETAILED DESCRIPTION OF THE INVENTION

A spinal implant fixation assembly constructed in accordance with the present invention is generally shown at 10 in FIG. 1. Similar structures amongst the several embodiments are shown by primed numbers in the various Figures.

More specifically, referring to the first embodiment of the present invention generally shown at 10 in FIG. 1, the assembly 10 includes a bone fixation element generally shown at 12 for fixation of the assembly 10 to a bone. A rod receiving mechanism is generally shown at 14 and is operatively connected to the bone fixation element 12. The rod receiving mechanism 14 includes a seat 16 having an inner wall 18 for seating a portion of a rod 20 therein. A locking mechanism generally shown at 22 engages the rod receiving mechanism 14 for forcing the inner wall 18 to contour around and engage the rod 20 seated therein and for locking and fixing the rod 20 relative to the assembly 10. In this manner, as the locking mechanism 22 forces the inner wall 18 to contour around and engage the rod 20 seated therein, there is increased surface to surface contact and therefore increased frictional engagement between the seat 16 and rod 20 thereby providing a more effective frictional contact. That is, the inner wall 18 of the seat 16 is compressed against the rod 20. The locking mechanism 22 is also seated against the rod 20. However, unlike prior art assemblies discussed above, the surface area engaging against the rod 20 is vastly increased over the prior art which increases the assembly to rod holding power.

More specifically, the rod receiving mechanism 14 includes a tapered outer surface 24. As shown in the several embodiments, this outer surface 24 is not threaded. Preferably, the locking mechanism 22 is in the form of a non-threaded cap 26 having an inner surface 28, which can be forced over and engage the outer surface 24 of the rod receiving mechanism 14. The non-threaded cap 26 inwardly deflects the rod receiving mechanism 14 about the seat portion 16 as the locking member 26 further engages the tapered outer surface 14.

Referring more specifically to the rod receiving mechanism 14, it includes a body portion 30 having two arms 32, 34 extending therefrom and being substantially parallel relative to each other. The two arms 32, 34 and the body portion 30 form a U-shaped inner surface defining the seat portion 16 thereof. Also, the arms 32,34 have the tapered surface 24 about the outer surface thereof. Thus, as the locking mechanism 22 in the form of the cap 26 is placed over the tapered outer surface 24 of the arms 32,34, the cap 26 compresses the arms 32,34 against a rod member 20 disposed within the seat 16. As stated above, this provides a vastly increased surface area engagement between the seating surface 16, inner walls 18 and rod member 20. The arms 32,34 provide for flexibility, yet are sufficiently rigid to maintain structural integrity.

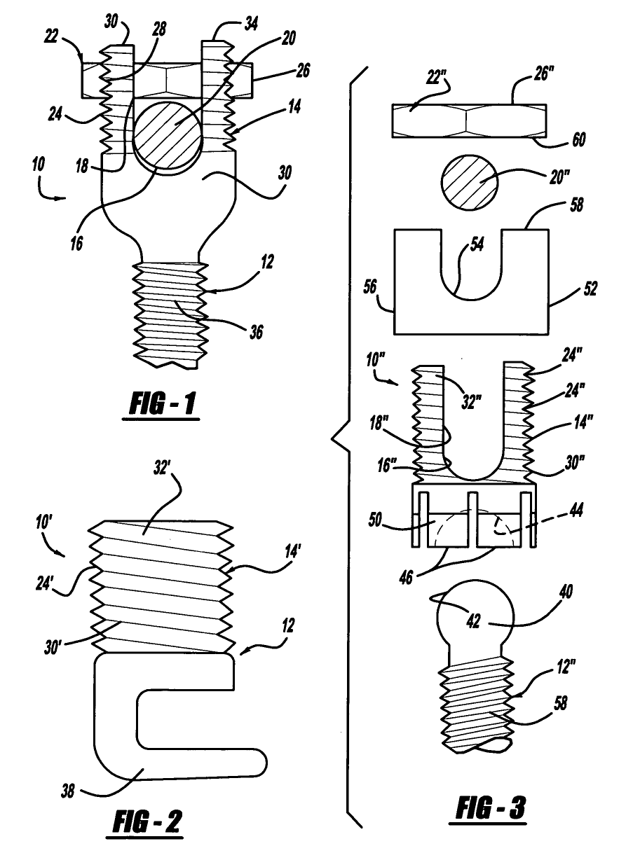

In the first embodiment shown in FIG. 1, the bone fixation mechanism 12 is shown as a screw portion 36 extending integrally from the body portion 30. The body portion 30 includes a longitudinal axis. The bone fixation element 12, whether it is a screw portion as shown in FIG. 1 at 36 or a hook portion 38 as shown in FIG. 2, can either 1) lie along the axis so as to define a substantially linear element or 2) be angled relative to the longitudinal axis of the body portion 30. In this manner, the device can be adapted to various angulations between the bone connection surface and the rod 20. These embodiments of the invention provide either a thread or hook portion 36,38, respectively, having the upper tapered threaded portion about the U-shaped seat 16. Variability of angulation is eliminated as each unit would be a solid fix piece. But the assemblies can be individually made in various angulations. Such assemblies provide solid fixation of implants to the rod 20 where angulation is either not required or where known angulation may be repeatedly needed.

As stated above, the bone fixation element 12 can take on various shapes and sizes known in the art. The element 12 can have various configurations as a screw 36 and various thread designs. Also, as shown in FIG. 2, the hook portion 38 can be manufactured and used in a variety of hook sizes. Other shapes and sizes well known in the art can also be used.

The assembly is preferably made from machined titanium or alloy, but can be alternatively made from other types of cast or molded materials well known in the art.

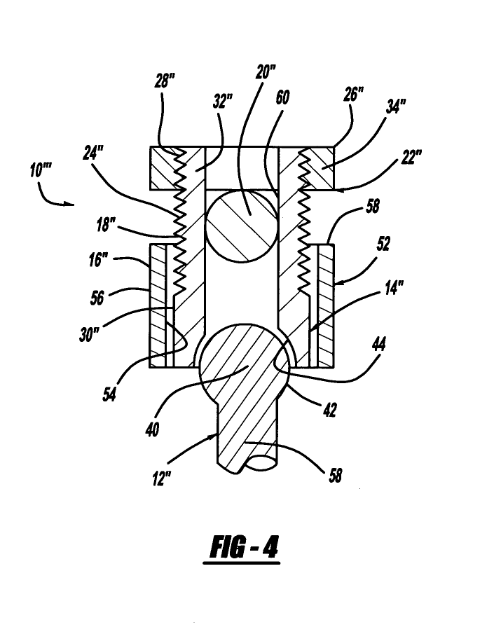

A second alternative embodiment of the present invention is shown in FIGS. 3 and 4. As stated above, double primed numbers are used to indicate like structure between the several embodiments.

Referring specifically to FIGS. 3 and 4, the bone fixation element 12″ is shown as an independent screw member. The element 12″ includes a head portion 40 having a substantially spherical outer surface 42. The rod receiving mechanism 14″ is shown as a single integral unit including the first seat 16″ for receiving the rod member 20″ as discussed above between the arms 32″ and 34″ and a second seating surface 44 having a substantially spherical shape for seating the head portion 40 of element 12″ therein.

Referring more specifically to the rod receiving member 14″, it consists of a substantially tubular body including the pair of spaced substantially parallel arms 32″,34″ extending therefrom and forming the substantially U-shaped seat 16″ as discussed above. The tubular body further includes a socket portion defining the second seat 44, which includes outwardly flaring flanges 46, as best shown in FIG. 3. The outwardly flaring flanges 46 have distal ends which flare radially outwardly relative to a central axis of the rod receiving member 14. The outer surfaces 50 define the outer surface of the second seat 44.

The locking mechanism 22″ of this embodiment includes the cap 26″ and a tubular sleeve member generally shown at 52. Although the cap 26″ and sleeve member 52 are shown as separate elements, the present invention could be practiced where the cap 26″ includes a skirt portion integrally extending therefrom. In either embodiment, the sleeve 52 locks and fixes the head portion 40 of the screw element 12″ within the seat 44 prior to the cap 26″ locking and fixing the rod 20″ within the seat 16″. The sleeve member 52 includes an inner surface 54 which, upon being disposed over and about the outer surface 50 of the flanges 46, engages and inwardly deflects the distally outwardly tapering surfaces thereof to engage the socket portion of the seat 44 with the head portion 40 of the screw member 12″. This can be accomplished prior to the connection of member 14″ with the rod 20″ and its locking in place by the cap 26″.

Referring more specifically to the sleeve member 52, it includes curved recessed portions 54 for seating of the rod member 20″ therein in the assembled configuration as shown in FIG. 4. The sleeve 52 also includes a skirt portion 56 which is disposed about the flanges 46 in the assembled position, as shown in FIG. 4. In the embodiment shown in FIGS. 3 and 4, the element 30″ includes the tapered outer surface 24″ which can be engaged by the inner surface 28″ of the cap 26″. As the cap 26″ is placed over the outer tapered surface 24″, it not only inwardly deflects the arms 32″,34″ to engage the rod member 20″ but also forces the skirt portion 56 of the sleeve member 52 over the outwardly flared flanges 46 so as to force the inner surface of the seat 44 to frictionally engage and hold in place in a fixed manner the head portion 40 of the screw element 12″. The screw element 12″ is then locked securely at whatever angle the components are in. This locking is independent of the locking of the rod 20″ in place.

This locking of the screw element can occur in two ways. The outer sleeve 52 can be pushed down with an instrument without the rod being in place or pushed down as the nut 26″ is tightened over the rod 20″. This gives the surgeon the option of adjusting the screw angle for abnormal anatomy and locking it prior to locking the rod 20″ to the assembly 10″ or, alternatively, locking the screw element 12″ and rod 20″ interfaces simultaneously when correction is not required.

As stated above, the head portion 40 is shown to be substantially spherical in shape. The seat 44 is a socket portion which is also substantially spherical for seating and engaging the head portion 40 therein. This allows for easy angular adjustment between the two components. Alternatively, the head portion 40 of the screw element 12″ can take on various other shapes, such as a square shape, which may not allow for similar angulation but would allow for similar connection between the head portion 40 and the seat 44 in accordance with the present invention.

In the embodiment as shown wherein the head portion 40 is of a spherical shape for mating with the spherically shaped female seating portion 44, the configuration allows for more of angulation in all directions relative to the shaft portion 58 of the screw element 12″. Thus, the present invention provides a multi-planar locking mechanism that allows for angulation in all planes. It also provides a locking mechanism that allows the mechanism to be locked at any angle prior to rod insertion. The invention also provides a multi-planar locking mechanism that reduces intraoperative rod contouring provides flexibility.

A further embodiment of the present invention is shown in FIGS. 8-11. This embodiment of the invention includes the bone fixation element generally shown at 12″″, this embodiment being characterized by including a screw head receiving insert generally shown at 90 which is moveable within the assembly 10″″ between a locked position as shown in FIGS. 10 and 11 entrapping the screw head 40″″ therein and an unlocked position wherein the screw head 40″″ enters or escapes, as shown in FIGS. 11 and 12. That is, this embodiment of the invention includes a single unit capable of receiving a screw head 40″″ therein and then allowing for polyaxial adjustment of the screw head relative to the assembly and then locking of the screw head within the assembly without requirement of additional elements to the assembly. This embodiment of the invention drastically reduces surgical time in spinal surgery and simplifies the elements needed for implementing the bone fixation. Such a system is particularly useful when the rod 20″″ in not lined up with the screw 12″″.

More specifically, the assembly 10″″ includes a body 30″″ including an internal portion 92″″. The internal portion 12″″ generally includes a first portion 94 which is radially outwardly recessed relative to a second internal portion 96. The internal portion 94 can be effectively recessed or actually recessed. The first portion could have a greater diameter than the second portion or the second portion could be formed by flanges that extend radially internally from an inner surface of the second portion thereby effectively defining the end of each flange as the radially inwardly extending surface.

The screw head receiving means 90 consists of a insert member 90 including a seat 44″″ for seating the screw head 40″″ therein. The insert 90 is moveable within the internal portion 92 between the locked and unlocked position as discussed below.

FIG. 12 shows an enlarged cross-sectional view of the insert 90 made in accordance with the present invention. The seat 44″″ more particularly includes a base portion 98 and a plurality of flexible arms 100 extending therefrom combining with the base portion 98 to form a pocket. The arms 100 define flexible walls of the pocket extending from the base portion 98.

As least one of the arms 100 includes a hinged portion 102 allowing for outward deflection of the arm 100. The hinged portion, as shown in FIG. 12, can be a recess cut into the base portion 98 adjacent the arm 100 to allow for increase outward flexibility of the arm 100, which includes the hinged portion 102. This allows for increased ease of insertion of the screw head 40″″ into the pocket.

FIGS. 8-11 sequentially show the method of using the present invention for fixing a polyaxial screw 12″″ therein. The screw itself 12″″ is inserted into the bone by itself. This provides excellent visualization of screw placement since the larger body/insert assembly 10″″ is pushed on the screw head after screw insertion into the bone.

As shown in FIG. 8, the insert 90 is sufficiently collapsible to be snapped into the internal portion 92 of the body element 34″″. This is accomplished by compressing the insert 90 and releasing it inside the internal portion 92. The assembly itself can be made from any durable material, such as carbon composites, nitinol, stainless steel, composite materials, plastics and plastic compositions or even resorbable materials. Preferably, titanium is used to minimize artifacts from x-rays and other diagnostic imaging systems. The combined assembly effectively provides the equivalent of a one piece assembly which is a significant improvement over prior art two piece assemblies or multiple piece assemblies necessary for only securing a screw head within a fixation device.

When the insert 7 is disposed within the first portion 94 of the internal portion 92, there is internal space to allow for slight expansion of the insert 90 therein. When the screw head 40″″ is disposed into the internal portion 92, the screw head 40″″ will effectively force the insert 90 into the first portion 94 thereby ensuring the ability of the pocket to expand sufficiently to allow insertion of the screw head 40″″ into the pocket. Once the screw head 40″″ is fully inserted into the pocket, the insert 90 snaps onto the screw head 40″″. In this condition, polyaxial movement can be achieved.

Locking can be achieved in two manners. The body 30″″ can be pulled up relative to the screw 12″″ with an instrument without the rod 20″″ being in placed or pulled by the nut 26″″ as the nut 26″″ is tightened over the rod 20″″. This provides the surgeon with the option of adjusting the screw angle for abnormal anatomy and locking it prior to locking the rod 20″″ to the assembly 10″″ or locking the screw 12″″ and rod 20″″ interfaces simultaneously when correction is not required.

As shown in FIG. 11, the U-shaped inner surface defining the seat portion 16″″ extends into the internal portion 92. Upon seating of the rod 20″″, the inserted portion of the rod 20″″, contacts a portion of the surface of the base portion 100 of the insert 90 for final seating of the insert 90 within the second portion 96 of the internal portion 92. As best shown in FIG. 13, which shows a cross section of the body portion 30″″, the second portion 96 includes a radially inwardly tapering surface. Thus, as the insert 90 is drawn into the second portion 96, the outer surface of the arms 100 of the insert 90 are progressively compressed about the screw head 40″″ thereby effectively engaging and locking the screw head 40″″ in position relative to the body portion 30″″. Upon final locking of the rod 20″″ within the assembly 10″″, as described above, complete fixation is achieved.

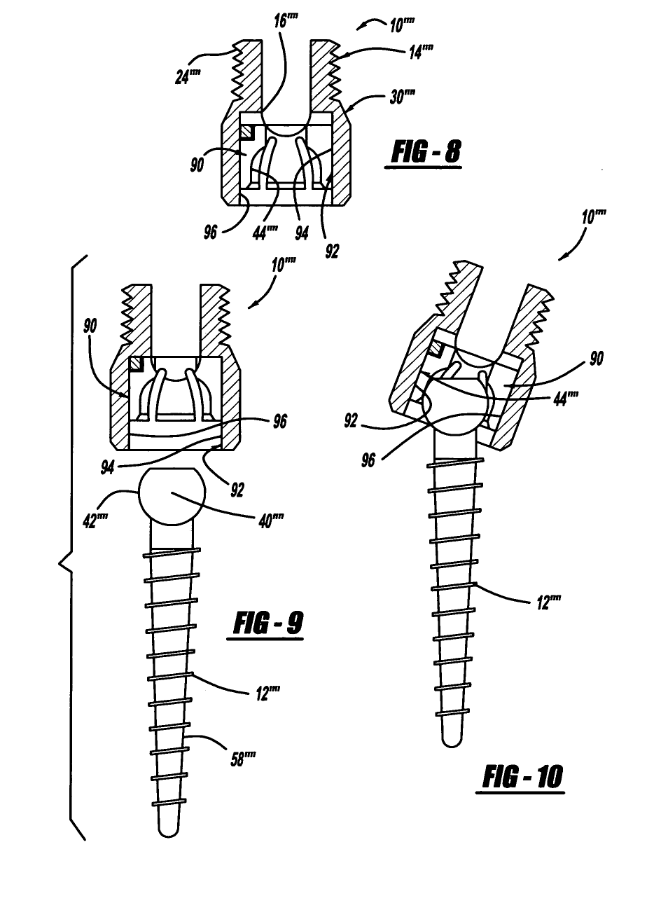

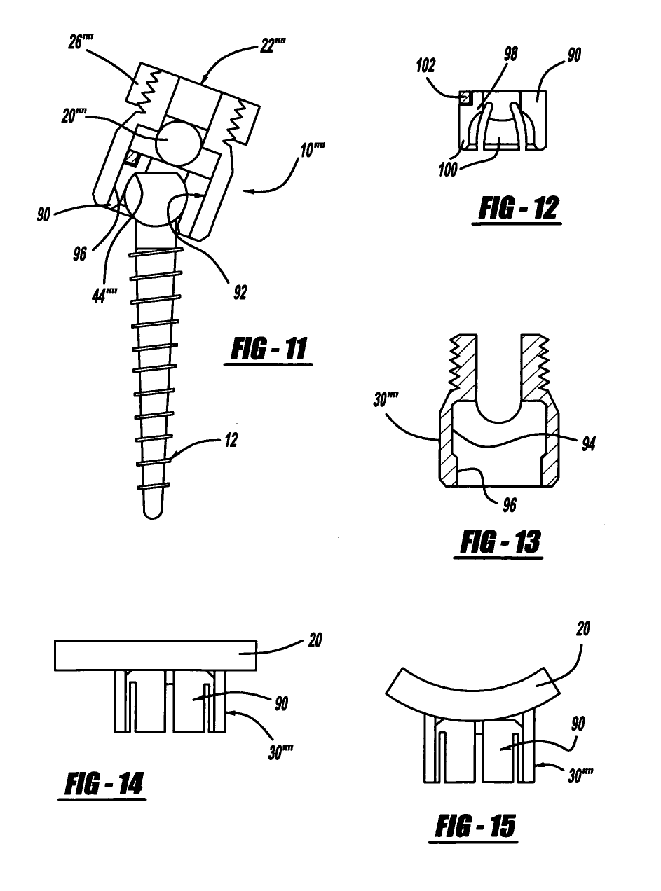

Also significant with regard to this embodiment is the fact that the nut 26″″, which includes a tapered threaded internal surface as discussed above, compresses the tapered threaded portion 14″″ of the assembly 10″″ against the rod 20″″. The nut 26″″ will also seat against the rod 20″″, but the surface area engaging the rod 20″″ will be vastly increased over the prior art, which increases the assembly to rod holding power. In fact, the nut against the rod is only a secondary locking means. The force of the portions 14″″ against the rod 20″″ is the primary locking mechanism. In other words, the rod 20″″ is engaged by the nut 26″″, the body portion 30″″, and the insert 90. Effective engagement of the insert 90 is significant as demonstrated in FIGS. 14 and 15.

FIG. 14 shows a cross section of the assembly wherein a straight rod 20 is retained within the assembly. With such a straight rod 20, the rod 20 will push the insert 90 down until the rod 20 fits within the U-shaped channel of the body 30″″. It is ideal for the rod 20 to contact the edges of the body 30″″ inside the U-shaped channel for maximum rod gripping strength. When the rod 20 is contoured, as shown in FIG. 15, the insert 90 of the present embodiment can self-adjust and be pushed downward further then the edges of the body 30″″ within the U-shaped cut-out to maximize rod contact. Such self adjustment is not at all found in the prior art since such U-shaped cut-outs in a body portion are fixed machine surfaces.

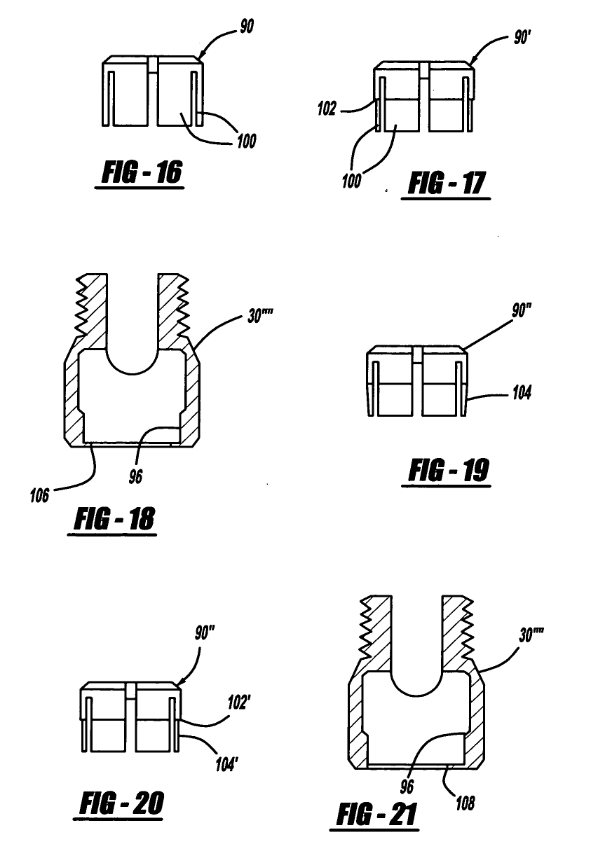

FIGS. 16-21 shown various permutations of the insert and body portions of the present invention. FIG. 16 shows an insert 90 including arms 100 having smooth outside surfaces. This is an embodiment that is shown in the previously discussed figures. In FIG. 17, the insert 90″ includes arms 100″ having an stepped outer surface 102. Such a step outer surface provides a stop for engaging the inner surface of the internal portion 92 to prevent the insert 90″ from moving beyond the desired engagement location. FIG. 19 shows an insert 90″ including a radially inwardly tapered outer surface portion 104 for progressive engagement with the second portion 92. FIG. 20 shows a further embodiment of the insert 90″ combining the inward tappered surface 104″ with the step 102″.

FIG. 18 shows a body portion 30″″ wherein the second portion 96 includes a radially inwardly extending lip 106 at the peripheral edge thereof. FIG. 21 shows a chamfered surface 108 at the peripheral edge of the second portion 96, both the lip 106 or the chamfered portion 108 provide further stops to ensure that once the insert member 90 is disposed within the internal portion 92, the insert 90 does not inadvertently exit therefrom.

The components for the assembly can be manufactured according to the following techniques, but every manufacturer has their own variations.

The body is made by first blanking the outer shape from round bar stock. By holding on the threaded end, or an extension to the threaded end (extra bar material), a hole is made into the opposite end. This hole is undersize relative to the taper to allow the taper to be but with a single tool. While the part turns in a lathe, a boring bar having a small cutting tip is introduced into the hole and the taper and recess cut. The threads are then cut, any extension cut off, and the slot either milled or cut by EDM.

The insert is made by cutting the outside cylindrical shape with an extension to hold on in a lathe. A hole is drilled into one end and a boring bar with a small cutting tip used to enter the hole and cut the spherical seat. The outer slots and hinge details are cut by either a slitting saw or a wire EDM.

Another possibility for the insert is to have a U cut or indentation in the top of it for seating of the rod. This is not preferable, since orientation of the insert would then be necessary, but possible.

Another addition to the body at the threaded portion is to add a recess in the side of the arms of the U on the inside for a rod to fit within. This would act as a guide for seating the nut with an instrument, as it would align the nut relative to the threads.

In combination, this last described embodiment provides a novel fixation assembly, which can be either combined with the novel rod retaining features described above or with other types of rod retaining features resulting in a simple effective and efficient means for fixing a screw member to a rod.

In accordance with this method, the locking mechanism is locked to the spherical head 40 of the bone fixation element 12″ at a desired angle prior to rod insertion or locked simultaneously by tightening of the nut member 26″. This locking method and the mechanism used therewith is fully reversible and top loading.

The cap 26 can be a nut, screw, set screw, or other similar cap 26 as is known to those skilled in the art. The locking mechanism 26 usually is threaded on at least one surface thereon. FIGS. 1, 3, 4, 5, 6, and 8-21 illustrate embodiments of the cap 26. As stated, the cap 26 is preferably non-threaded. The cap 26″ can include a non-threaded exterior surface 60. The cap 26″ includes a body 61 having a top portion 62 and a bottom portion 64. Preferably, both the top portion 62 and the bottom portion 64 have radially, unthreaded exterior surfaces 60. The cap 26″ can also include an engagement portion 76, on the bottom portion 64 of the cap 26″, for engaging the fixing mechanism 18 of the assembly 10. The engagement portion 76 ensures that the cap 26″ maintains proper positioning within the assembly 10 and is fixed relative thereto. The engagement portion 76 can be a tab 78 that mates with a groove 80 on fixing mechanism 18. The fixing mechanism 18 is preferably formed of a flexible material such that it allows radially extending frustoconical movement, thereby enabling easier insertion of the cap 26′. Alternatively, the engagement portion 76 can be a tab 82 on the fixing mechanism 18 that mates with a groove 84 located within the engagement portion 76. In either instance, the tab 80, 82 is any form of protrusion that can be maintained within a groove 78, 84 via an interference fit. For example, the tab can be a circumferential tab on the exterior surface 60 of the cap 26″. The tab can be on a portion of the exterior surface 60 of the cap 26″ or completely surround the exterior surface 60 of the cap 26″.

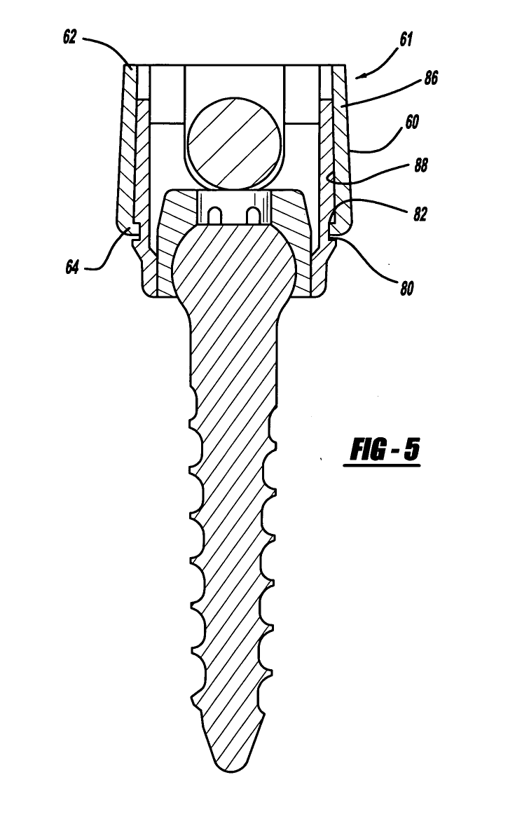

The cap 26″ can be a tapered locking cap 86 as shown in FIGS. 5 and 6. The tapered locking cap 86 is preferably a self-locking taper that fits a mating taper 88 on the exterior surface 88 of the fixing mechanism 18. In other words, the exterior surface 88 of the fixing means 18 is tapered as is the tapered locking cap 86 such that the two pieces mate and lock together to maintain the assembly 10 in proper alignment. The tapered locking cap 86 can also include an engagement portion 76″. The engagement portion 76″ is a device then ensures that the tapered locking cap 86, and thus the cap 26′″, maintains proper positioning within the assembly 10. The engagement portion 76″ can be a groove 78″ that mates with a tab 80″ on fixing mechanism 18. Alternatively, the engagement portion 76″ can be a tab 82″ on the engagement portion 76″ that mates with a groove 84″ located within the fixing mechanism 18. In either instance, the tab 80″, 82″ is any form of protrusion that can be maintained within a groove 78″, 84″ via an interference fit. For example, the tab 80″, 82″ can be a circumferential tab on the exterior surface 60″ of the cap 26′″. The tapered locking cap 86 can include an aperture 70″ on a top 72″ of the top portion 62″ of the cap 26′″. The aperture 70″ can be used for the insertion and removal of the cap 26′″. The aperture 70″ can include threads 74″ or other mechanisms that are beneficial for inserting and removing the cap 26′″. Alternatively, the aperture 70″ can be shaped such that a specific tool can be used for inserting and removing the cap 26′″.

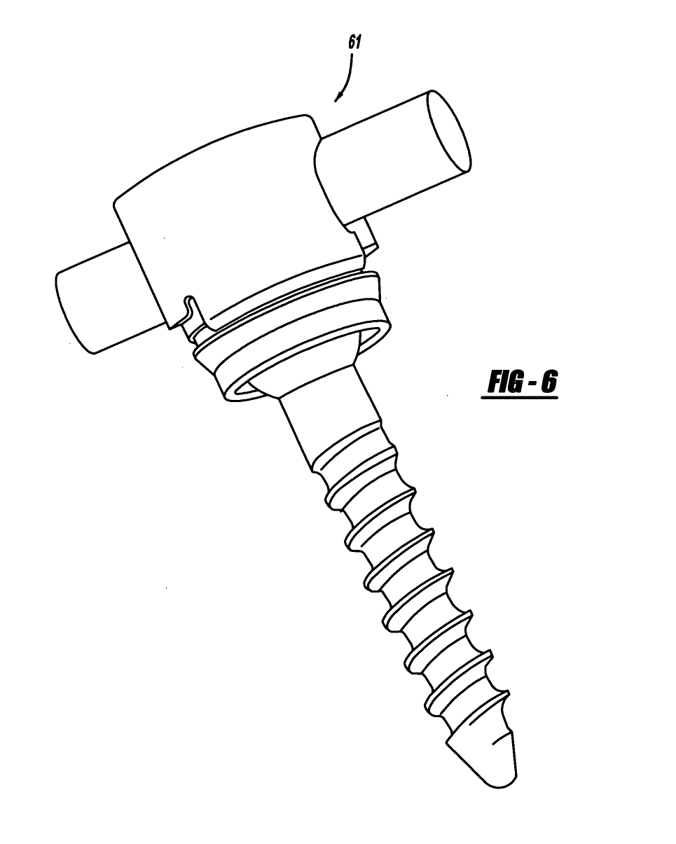

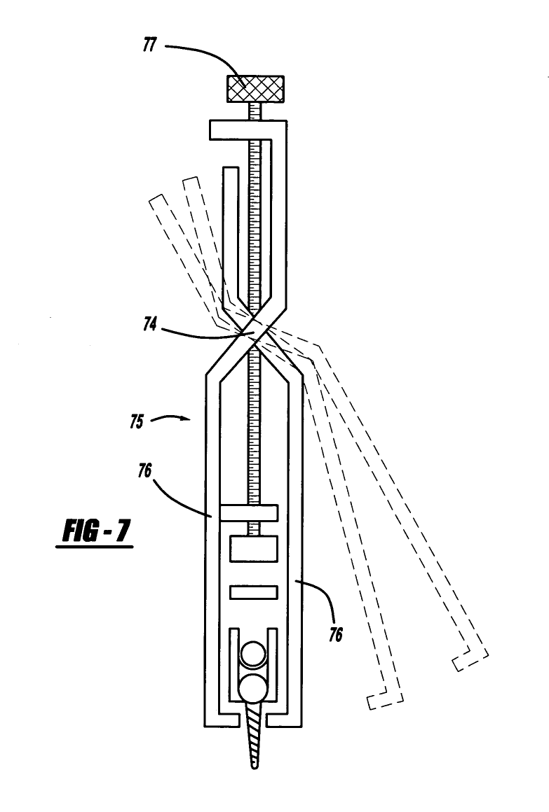

For inserting the locking mechanism of the present invention numerous tools known to those of skill in the art can be used. An example of one such tool is shown in FIG. 7. The insertion device 75 includes arms 76 that clampingly hold the assembly 10. The arms 76 are z-shaped, in this embodiment, such that they cross over one another, as shown in the figure. Other arms 76, embodiments, or examples of similar devices can also be used as long as the devices can clamp together in a parallel clamping fashion. At the cross-over junction 78, there is a device that holds the assembly 10 in position within the insertion device 75. A knob 77 is affixed to the holding device. The knob 77 includes an internal lock for maintaining the assembly 10 in position. The knob 77 can effectively function as a plunger and force the assembly 10 in place while preventing torsional forces from being translated to implants and bone during assembly.

With more specific regard to the locking mechanism, the sleeve ring 52 includes an edge surface 58. The cap 26″ includes an abutment surface 60 for abutting against the edge 58 as the cap 26″ is placed onto the tapered portion 24″ to force the ring member 52 over the outer surface of the flanges 50.

In operation, the screw element 12″ is fixed onto a bone, the head portion 40 extending from the bone surface. The rod seating member 14″ is then disposed over the head portion 40 of the screw element 12″ by insertion of the head portion 40 into the seat 44. This is a snapping operation but allows for angular adjustment of the tubular member 14″ relative to the longitudinal axis of the screw element 12.″. The ring 52 is then disposed over the member 14″ and an instrument is used to force the ring member 52 over the flanges 50 so as to lock the head portion 40 within the seat 44 thereby fixing the angulation between the two elements. The rod 20″ is then seated within seat 16″ of the member 14″ as well as within the groove 54 of the ring 52. Finally, the cap 26″ is placed over the tapered outer surface 24″ of the arms 32″,34″ thereby fixing the rod 20″ in frictional engagement within the seat 16″ and against the cap 26″. Alternatively, as discussed above, the cap 26″ can be used to force the sleeve member 52 in place so as to lock the head 40 and screw member 12″ relative to the element 14″.

Utilizing the embodiment of the present invention as shown in FIGS. 1 and 2, the process is exactly the same with regard to locking the rod member 20 in place once the screw or hook portions 36,38, respectively, are connected to the known.

In view of the above, the present invention provides a method for locking a rod 20, 20″ to a bone by the general steps of first fixing a rod seating member 14,14′,14″ to a bone and then seating a portion of the rod 20,20″ within a substantially U-shaped seat 16,16″ of the seating member 14,14′,14″. The rod 20,20″ is locked within the U-shaped seat 16,16″ while engaging and contouring at least a portion of the U-shaped seat 16,16″ about the rod 20,20″. As shown in FIGS. 3 and 4, this method can be more specifically defined by the steps of fixing the bone fixation member 12″ to a bone and then locking and fixing the rod seating member 14″ to the head portion 40 of the bone fixation member 12″ and then locking the rod 20″ within the U-shaped seat 16″. The fixing step is accomplished by forcing the ring 52 over the outwardly flared portions 46 of the seat portion 44 to lock and fix the head portion 40 of the bone fixation element 12″ therein. Finally, the locking of the rod is accomplished by locking the rod 20″ within the U-shaped seat 16″ by engaging the inner surface 28″ of the cap 26″ over the tapered outer surface 24″ of the U-shaped seat 16″ to force the ring 52 over the outer surface 50 of the seat portion 44 to lock and fix the head portion 40 of the bone fixation element 12″ therein while simultaneously deforming the inner surface of the U-shaped seat 16″ about the rod 20″ seated therein.

In accordance with this method, the locking mechanism is locked to the spherical head 40 of the bone fixation element 12″ at a desired angle prior to rod insertion or locked simultaneously by tightening of the cap 26″. This locking method and the mechanism used therewith is fully reversible and top loading.

The cap 26 secures and tightens the entire screw and rod fixation assembly 10. The cap 26 includes a deflecting mechanism 38 for deflecting at least one flexible portion 46 of the rod seating mechanism 28 against and around the rod 16 as said cap 26 further engages the at least one flexible portion 46 of the rod seating mechanism 28. The deflecting mechanism 38 can be an inner surface or portion of the cap 26 itself. The deflecting mechanism 38 further engages the tapered outer surface 34 therein.

The components for the screw fixation assembly and device disclosed and described herein can be manufactured by various methods known to those of skill in the art.

Throughout this application various publications are referenced by author and year. United States patents however, are referenced by number and inventor. Full citations for the publications are listed below. The disclosures of these publications and patents in their entireties are hereby incorporated by reference into this application to more fully describe the state of the art to which this invention pertains.

The invention has been described in an illustrative manner, and it is to be understood that the terminology that has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A locking mechanism comprising non-threaded locking means for locking a rod in place within a screw and rod fixation assembly.

2. The locking mechanism according to claim 1, wherein said locking means is tapered.

3. The locking mechanism according to claim 2, wherein said tapered locking means includes an automatic locking taper for mating with an outer surface of a rod locking device of the screw and rod fixation assembly.

4. The locking mechanism according to claim 1, wherein said locking means includes engagement means for engaging an aperture in the screw and rod fixation assembly.

5. The locking mechanism according to claim 4, wherein said engagement means is selected from the group consisting essentially of a groove, a tab, and a snug fit.

6. The locking mechanism according to claim 1, wherein said engagement means is a single tab.

7. The locking mechanism according to claim 1, wherein said engagement means is at least two tabs.

8. The locking mechanism according to claim 1, wherein said tabs are positioned at least 90 degrees apart from one another.

9. The locking mechanism according to claim 1, wherein said tabs are positioned at least 180 degrees apart from one another.

10. The locking mechanism according to claim 1, further including an insertion device for inserting the locking mechanism.

11. A method for locking a bone fixation assembly in place by:
engaging a non-threaded locking mechanism with a rod receiving mechanism of a bone fixation assembly, thereby locking the bone fixation assembly.

12. The method according to claim 11, wherein said engaging step included contouring the non-threaded locking mechanism around the rod receiving mechanism.

13. A locking mechanism insertion device comprising:
at least two clamping arms, each having an insertion end for holding a locking mechanism and a distal end; and
operating means on said distal end of said arms for operating said device.

14. The locking mechanism insertion device according to claim 13, wherein said operating means is a knob.

15. The locking mechanism insertion device according to claim 14, wherein said knob included locking means for locking said device in place.

16. The locking mechanism insertion device according to claim 13, wherein said arms are z-shaped.
